(19) United States
(12) Patent Application Publication
(10) Pub. No.: US 2016/0000403 A1
(43) Pub. Date: Jan. 7, 2016

(54) Method and Apparatus for Monitoring Cardiac Output

(71) Applicant: DVX, LLC, Princeton, NJ (US)

(72) Inventor: David Vilkomerson, Princeton, NJ (US)

(21) Appl. No.: 14/753,056

(22) Filed: Jun. 29, 2015

Related U.S. Application Data
(60) Provisional application No. 62/020,917, filed on Jul. 3, 2014.

(51) Int. Cl. A61B 8/12; A61B 8/00; A61B 8/06

(52) U.S. Cl. CPC A61B 8/12; A61B 8/065; A61B 8/4477

(57) ABSTRACT

A new method of measuring the cardiac output is disclosed. The new method uses an ultrasound emitter and one or more receivers placed in the superior vena cava just above the right atrium of the heart so that the ultrasound apparatus can transmit through the wall of the superior vena cava and the juxtaposed wall of the aorta at this location. By measuring the velocity of the blood by its back-scattered Doppler shift, the cardiac output can be determined. The volume of blood flow can also be determined by measuring the diameter of the aorta.

### CROSS-REFERENCE TO RELATED APPLICATIONS

This application claims the benefit of U.S. Provisional Application Ser. No. 62/020,917, filed on Jul. 3, 2014. The disclosure of the above application is incorporated herein by reference in its entirety for any purpose.

### FIELD OF THE INVENTION

The present invention generally relates to the monitoring of blood flow out of the heart, “cardiac output”, and more particularly to the measurement of velocity and volume of blood flow out of the heart using Doppler techniques.

### BACKGROUND OF THE INVENTION

As life depends upon the heart pumping blood through the cardiovascular system, monitoring the blood coming out of the heart, cardiac output, is a major concern to physicians. While there are indirect indications of cardiac output, including skin appearance and blood pressure, various devices are presently used to determine cardiac output, and in particular, changes in cardiac output. As the absolute measure of liters/minute of cardiac output depends on body size and activity, what physicians are most concerned with is seeing changes in cardiac output as they change conditions, e.g. adding fluid to the blood or using various drugs to increase the strength of the heart's beating or constrict the peripheral parts of the cardiovascular system.

There are a number of methods and devices used to perform cardiac output measurement. These include inserting a catheter into the right side of the heart, with the end of the catheter in the output vessel of the right side of the heart, the pulmonary artery, to measure the cardiac output of the right side of the heart. When a known amount of chilled portion (“a bolus”) of saline fluid is injected into the entrance of the heart via an exit from the catheter at that position, the rate of temperature change of the blood measured at the outflow of the right heart into the pulmonary artery shows how much total blood is being pumped through the right side of the heart. This equals the cardiac output unless there is bleeding in the lungs. This is known as the “thermal-dilution” method of measuring blood flow, and the specialized catheter used as a pulmonary catheter is known as a Swan-Ganz catheter. It is estimated that about a million such catheters are used each year for cardiac output measurement. However, modern guidelines suggest the use of the Swan-Ganz only for high-risk patients. For such patients the importance of hemodynamic information provided by this monitoring method, particularly for cardiac output, outweighs the risks imposed by inserting a catheter into the beating heart.

There are other less invasive means of monitoring the cardiac output. These include measuring the change in electrical impedance across the chest caused by the pulsing blood flow out of the heart (e.g. “BioZ” by CardioMetrics), measuring pressure waveforms (e.g. “Flo-Trac” Edwards), or directly measuring the blood flow through the descending aorta (˜80% of the total cardiac output) by Doppler ultrasound measuring apparatus located inside esophagus adjacent to the descending aorta, such as U.S. Pat. No. 4,796,634 or “CardioQ” from Deltex Medical.

In terms of monitoring, all these methods have deficiencies: measurement from inside the esophagus cannot be used for the hours and days needed for monitoring; unstable patients (e.g. those just back from cardiac surgery or infused with inotropes or vasopressors) do not fit the models of circulation on which systems such as the FlowTrac are based; edema in such patients makes unreliable cardiac output measurements based on the electrical impedance across the chest. Hence there is a need for an accurate method of monitoring cardiac output over extended time periods.

### SUMMARY OF THE INVENTION

The present invention discloses method of measuring the cardiac output. The new method utilizes the unique anatomical location where the superior vena cave (SVC) is juxtaposed to the ascending aorta, which carries the cardiac output. In this position just above the right atrium, Doppler ultrasound apparatus is placed in the SVC so that it can transmit ultrasound through the walls of the SVC and aorta and by measuring the Doppler shift caused by the blood flow in the aorta determine the cardiac output flow. As this location is where “central-line catheters” are conventionally placed to infuse fluids and drugs into the circulation of a patient and remain, sometimes for weeks at a time, monitoring by this new method can last an extended period of time—a very important benefit.

### BRIEF DESCRIPTION OF THE DRAWINGS

FIG. 1 is a line tracing from Gray's Anatomy to illustrate relevant parts of the heart and the blood flow monitoring method according to one aspect of the present invention.

FIG. 2 is a schematic representation of a conventional pulse-echo Doppler apparatus inside the superior vena cava (SVC) at the juxtaposition of the SVC and the ascending aorta according to one aspect of the present invention.

FIG. 3A shows a diffraction-grating-slab Doppler ultrasound film flow-measuring apparatus mounted on a cylindrical central line at the area of juxtaposition of the SVC and aorta according to one aspect of the present invention.

FIG. 3B shows a cross section of configuration shown in FIG. 3A according to one aspect of the invention.

FIG. 4 shows the shaped end of a catheter holding the flowsensing transducer array “sail” to the optimal location pushed by the blood flowing down the SVC according to one aspect of the present invention.

### DETAILED DESCRIPTION OF THE PREFERRED EMBODIMENTS

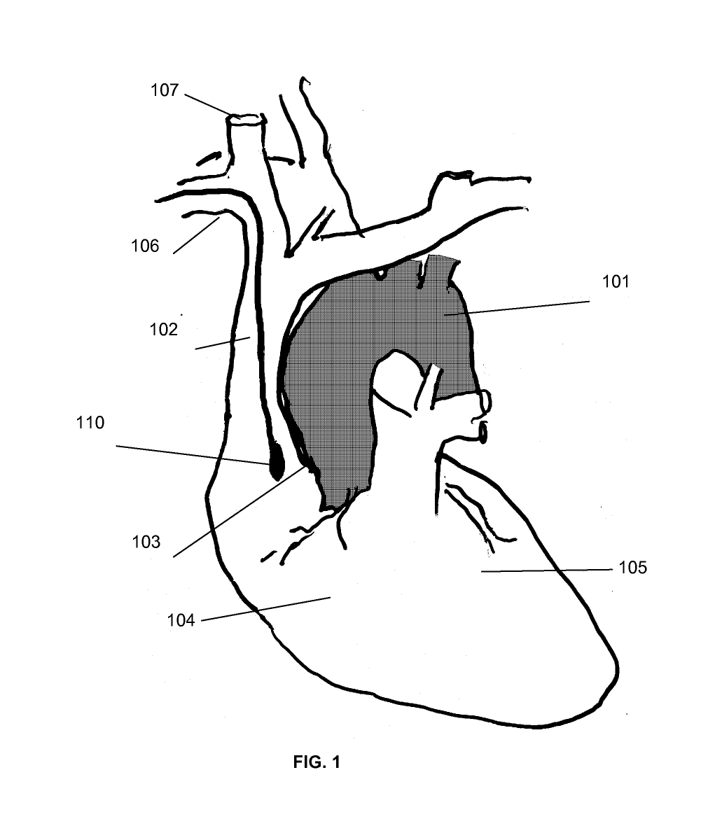

With reference to FIG. 1, the blood pumped out of the heart flows directly through the aorta (101) at high pressure. On the return path, the blood flows in lower pressure and returns to the heart through the superior vena cava, SVC (102). According to the present invention, the region for measuring the blood flow would be the juxtaposition of the superior vena cava and the aorta (103) where it is feasible to place a carrying structure, e.g. a thin flexible catheter (102) for carrying the Doppler apparatus near its tip (110) to this desired monitoring location (103). Relevant parts are also shown in FIG. 1: the right ventricle (104), the left ventricle (105), the entrances to the SVC, the subclavian vein (106) and internal jugular vein (107) that can be used for placing such carrying structures.

According to one aspect of the present invention, Doppler ultrasound is used at a particular anatomical area to measure the amount of blood pumped by the heart. This can be accomplished by positioning a pulsed Doppler system in the region near the entrance to the right atrium, as shown in FIG. 1. Methods of measuring blood flow by means of Doppler ultrasound in general are well known; see for example Chapter 12, Volumetric Blood Flow Measurement, Evans and McDicken, Doppler Ultrasound, 2^{nd} Edition, John Wiley & Sons, Chichester (2000).

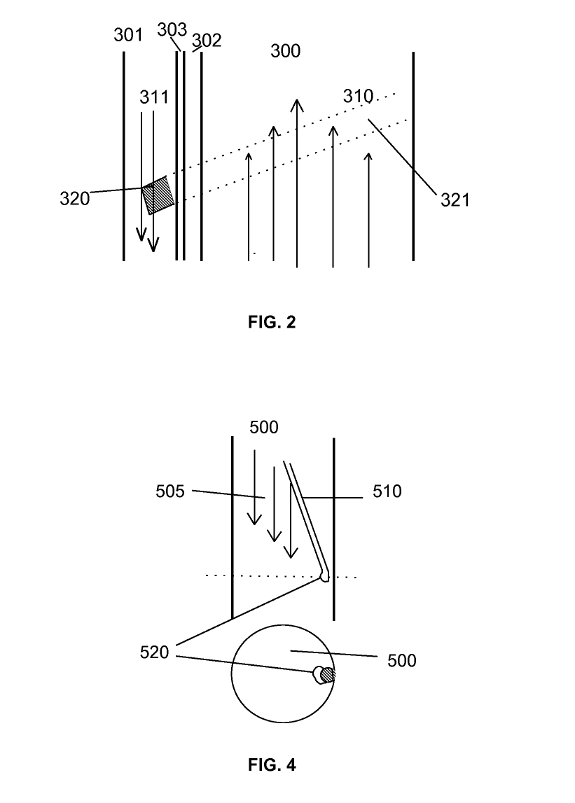

With reference to FIG. 2, the excited ultrasound pulse by the transducer (320) is transmitted following the path (321) that is perpendicular to the transducer (320), and passes through the wall (303) of the superior vena cava (301) and the wall (302) of the aorta (300) at where it is juxtaposed to the wall of the superior vena cava (302), and scatters from the moving blood red blood cells (310) of the blood in the aorta. Normally, the wall (303) of the SVC is less than 1 mm thick and the wall (302) of the aorta is less than 3 mm thick.

The movement of the scattering blood cells upward through the aorta Doppler shifts the frequency downward. The scattered ultrasound passes back through the wall of the aorta and the wall of the superior vena cava, and the now receiving transducer converts the ultrasound energy to electrical energy, which can be processed, as is well-known and practiced, into a Doppler electrical signal. The Doppler shift in frequency is proportional to the velocity of the scattering blood cells, and by integrating the ultrasound power at different frequencies in the spectrum of the Doppler signal the average velocity, which is proportional to the flow, can be ascertained, as is well-known to those skilled in the art. As the blood flow in the aorta (310) is moving away from the heart, and the blood flow in the SVC (311) is moving towards the heart, the Doppler frequency shifts generated from the opposite flows in the SVC and the aorta are opposite, and the negative Doppler shifts of the signals from the aorta are easily separated from the positive Doppler shifts arising from the flow in the vena cava.

According to one aspect of the present invention, the frequency of operation of a Doppler system is determined, as is well known by skilled artisans, by recognizing that the Doppler signal from the scattered blood increases as frequency to the fourth power and decreases due to the attenuation of any tissue the ultrasound traverses, at an attenuation of about 1 dB/cm-MHz. For traversing the walls of the vena cava and the aorta, 20 MHz is a desirable frequency for maximizing the signal for the average size individual; it may be necessary to adjust the frequency for different size individuals, e.g. children.

The Doppler apparatus must be small enough to fit inside the superior vena cava, often less than a centimeter in diameter, without blocking it: the superior vena cava is the return conduit for all the blood flow in the head and upper portion of the body. This is difficult with conventional Doppler ultrasound apparatus. Therefore, according to one aspect of the present invention, a thin film Doppler ultrasound apparatus that utilizes a diffraction-grating transducer (“DGT”) and a “film-slab” transducer adjacent to it to measure blood flow in a vessel, is used. This Doppler apparatus is described in detail in U.S. Pat. No. 7,963,920 ('920 patent) to Vilkomerson et al, which is incorporated herein by reference.

### An Exemplary Embodiment

The '920 patent solves the problem of a Doppler transducer impeding the flow in a vessel. The key difference in the flowsensor apparatus disclosed in '920 patent from conventional Doppler apparatus is that the ultrasound energy is launched at an angle to its surface, whereas the conventional transducers produce their beams perpendicularly to their surface. As the Doppler shift in frequency is Δf, the velocity of the flow v is represented by Δf=2×cos θv/λ, where θ is the angle between the flow vector and the beam vector, and A the wavelength of the ultrasound. In a conventional Doppler apparatus, if the surface of the transducer is parallel to the vessel wall, that would produce a beam at right angles to the velocity of the blood to be measured, and there would be no Doppler shift because the cosine of the 90° is zero. The DGT transducer in '920, however, transmits ultrasound at an angle (with respect to the perpendicular line to its surface) given by sin θ=λ/d, where d is the periodicity of the elements, as described in detail in '920.

According to one aspect of the present invention, the DGT transducer in '920 is employed in monitoring the blood flow in the aorta. The Doppler apparatus (as described in '920 a film flexible transducer) is wrapped around at or near the tip of a conventional 6-8 French (2-3 mm in diameter) “central venous line,” which is a catheter commonly used for purposes of providing a rapid access into the body for drugs and nourishment. This type of catheter is placed into the superior vena cava at the cavoatrial region, where the Doppler system is to be placed for cardiac output measurement. The region of juxtaposition through which the Doppler measurement takes place is approximately 4 cm in length. However, this dimension may vary depending on body size, so, for example, for children a different design may be optimal. The fabrication of such flexible film transducers has been disclosed in “Development of a Flexible Implantable Sensor for Postoperative Monitoring of Blood Flow” by Cannata et al, Journal of Ultrasound in Medicine 2012; vol 31; 1795-1802.

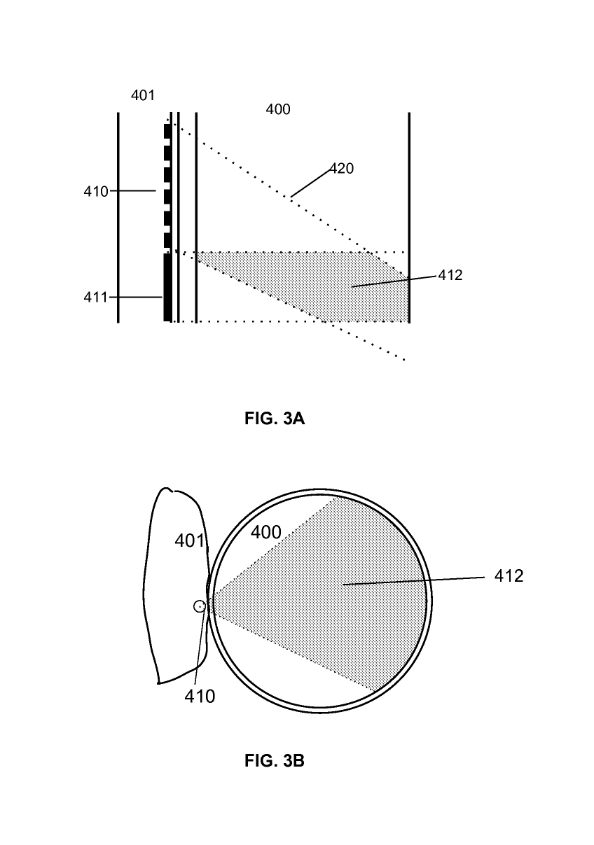

With reference to FIG. 3A, the diffraction-grating transmitter (410) and the slab transducer (described in detail in '920) (411) are situated longitudinally along the near wall of the vena cava. The ultrasound beam (420) is crossing the receiving beam of the slab transducer (411), to create a “sensitive region” (412), i.e. where any moving blood cell is insonated by the transmitting DGT's beam, and its scattered ultrasound is received by the slab transducer. At 20 MHz, the finger structure of the DGT (410) to produce a beam at a 33-degree angle is, according to '920, 34 micron wide fingers separated by 35 microns. As described in '920, the piezoelectric material composing the DGT and slab is P(VDF₇₅-TrFE₂₅) 25 microns in thickness. If the transducer is directly on the wall, the slab and dgt are placed directly next to each other; if, as described later, the transducers are not on the wall, the slab transducer is ideally longitudinally situated on the carrying-structure at a distance from the dgt equal to the tangent of the angle of the beam, (in example above for 33 degrees, 0.65) multiplied by the perpendicular distance of the carrying structure to the near aortic wall.

According to one aspect of the present invention, the placement of the catheter will not require exact positioning. As long as the sensitive area (430) is large enough to intercept the peak velocity of the blood flow, which is close to the center of the aorta, the peak velocity of the blood flow can be accurately determined, even if the transducer is not on the SVC wall. The size of the sensitive region can be adjusted by the design of the flow sensor, for example, longer transducers and lower beam angles make a bigger sensitive area.

With reference to FIG. 3B, according to another aspect of the present invention, the DGT transducer (410) is curved radially (as it is wrapped around the catheter) with respect to the center of the aorta and facing to the wall of the aorta juxtaposed to the wall of the SVC so that the excited ultrasound beam covers a portion of the lumen of the aorta, allowing the ultrasound to be scattered by a majority of the blood flow in the aorta.

The signals detected by the receiver transducer is amplified, heterodyne-detected, and the resulting Doppler power spectrum obtained by FFT processing. (Typical circuits to perform these processes are described in detail in the above-mentioned Evans and McDicken.) The Doppler power spectrum can be analyzed to determine the peak velocity, as taught, for example, in “Finding the Peak Velocity in a Flow from Its Doppler Spectrum” by Vilkomerson et al, IEEE Transactions on Ultrasonics, Ferroelectrics, and Frequency Control, vol. 60, no. 10, October 2013, page 2079.

According to one aspect of the present invention, as disclosed in '920 patent, the DGT transducer and slab transducer can be interchanged under the principle of reciprocity. For example, the DGT transducer can be a receiving transducer while the slab transducer is an emitting transducer.

As the Doppler signal varies with the angle of insonation and reception, it is possible that changes in body position could change the orientation of the Doppler flow-measuring system, therefore changing 8 and falsely indicating a change in cardiac output. As taught in “Diffractive Transducers for Angle-Independent Velocity Measurements” by Vilkomerson et al, Proc. of 1994 International Ultrasonics Symposium, IEEE Press, Piscataway (1995) pp 1677-1681, changing the frequency of the ultrasound and measuring the change in Doppler shift allows the angle of the flow vector to the flowsensor to be determined absolutely. As well known, if there are two Doppler equations in two unknown variables, velocity and angle, solving the two equations simultaneously yields both the unknowns, velocity and angle. According to one aspect of the present invention, the Doppler transducer as shown in FIGS. 3A and 3B is operated at least two different frequencies to take multiple Doppler measurements, so that the angle of the apparatus to the flow can be determined and the measurement compensated for the change in orientation. For example, if the catheter were at an angle to the flow of a and of velocity v, the Doppler-shift frequencies would be different:

f_{d1}=cos(φ+α)(−v)/λ and f_{d2}=cos(φ−α)v/λ

Solving simultaneously these two Doppler equations allows calculation of the angle of the catheter to the flow α, as well as the velocity v.

According to another aspect of the present invention, a DGT transducer with two receivers that are placed adjacent to each side of the DGT can be used to provide an angle-independent velocity determination. The two-receiver transducer is taught in “Double Beam Diffraction-Grating Transducers for Improved Blood Flow Measurement” by Vilkomerson, Proc. of 2008 IEEE International Ultrasonics Symposium, IEEE Press, Piscataway (2009) pp 1064-1067.

The double-beam DGT produces two beams at opposite and equal angles to the face of the double-beam DGT; if the double-beam DGT is exactly parallel to the flow, this would produce Doppler shifts of equal magnitude but opposite in sign in the two adjacent receivers. However, if the DGT and receivers are not parallel to the flow, two different Doppler frequencies will be produced. With two Doppler equations in the two unknowns of velocity and angle of flow with respect to the DGT solving them simultaneously will yield both velocity and angle of flow with respect to the transducer.

While relative changes in the cardiac output are most important clinically, if the absolute volume of blood flow is of interest, the area of the aorta as well as the flow velocity must be measured. According to one aspect of the present invention, pulse excitation of the slab transducer (411) will produce a pulse that will reflect from the two walls of the aorta, and, as is well known, the time between the reflections can be converted into distance by multiplying by the velocity of sound in blood, 1564 m/sec. If, as shown in FIG. 3, the slab is parallel to the wall, the diameter is equal to that distance, and because the aorta is substantially circular, the diameter will yield the area of the aorta by using the diameter squared multiplied by it divided by 4. The product of the average velocity and the area will be the flow volume.

If, however, the apparatus is not parallel to the walls of the aorta, the flow volume can be calculated because the angle of the apparatus to the aorta can be determined by the methods described above, as the flow is parallel to the walls of the aorta. The true diameter is calculated from the pulse-echo method above by multiplying by cosine of the angle of the apparatus to the aortic walls. Thus, the true volume can be determined if the apparatus is moving or not parallel to the SVC or aortic walls.

According to another aspect of the present invention, the flowsensor is stabilized in the vena cava so changes in angle do not occur. This is achieved by utilizing the blood flowing in the vena cava to stabilize the carrying structure's position. With reference to FIG. 4, by placing a curved section (520), on or near the end of the carrying structure (510), the blood flow (505) in the vena cava (500) forces the curved tip (520) that carries the Doppler apparatus to the wall of the vena cava nearest the aorta. Another method of orienting the apparatus is to use mechanical means such as expandable mechanical devices, e.g. a structure similar to a retrievable Greenfield filter that are often used in the inferior vena cava.

The examples and disclosures herein are not meant to be exhaustive but rather to indicate the different ways those skilled in the art will be able to utilize the present invention to make accurate measurement of cardiac output. For example, the present invention can be not only applied to human but also adapted to the measurement of cardiac output of an animal, such as a pig's heart. Further, the DGT film transducer in '920 is only exemplary, whereas other Doppler apparatus can also be utilized and adapted to be placed at a juxtaposition of SVC and the aorta inside SVC as long as it can intercept the peak velocity of blood flow in the aorta.

Still further, the conventional pulse-echo catheter-based Doppler system that exist (e.g. the AcuNav by Siemens) could use the method disclosed in the present invention. As shown in FIG. 2, a conventional Doppler system can be placed at the special cavoatrial position and used in a single or double mode. In the single mode, the transducer first transmits a signal and then receives the Doppler-shifted ultrasound signal. If absolute values of velocity are desired, the transducer can work in a double mode—it produces at least two beams at different angles, either mechanically or, electronically steered (e.g. beam steering in a phased-array like the AcuNav), and using the methods described above, the absolute blood velocity and flow angle (i.e. the angle of the velocity with respect to the measuring ultrasound beam) can be determined via solving the resulting two Doppler equations for those variables. Hence, the absolute velocity is determined.

Still further, DGT and the slab transducers are generally situated longitudinally but the distance between them can vary. Although the DGT and the slab transducers are situated adjacent to each other in the exemplary embodiment of the present invention, the ideal distance between the two transducers depends upon the angle and the distance between the transducers and the near wall of the aorta (e.g., the distance for pediatric use would be different than that for adult use). Similarly, if a mechanical device is used to hold the transducers the optimal distance between the two transducers will vary with the position of the transducers relative to the aortic wall.

Still further, the fabrication of Doppler transducer can be known method such as disclosed in “Development of a Flexible Implantable Sensor for Postoperative Monitoring of Blood Flow” by Cannata et al, Journal of Ultrasound in Medicine 2012, vol 31, pp 1795-1802, or any other methods or any after-arising fabricating methods.

The carrying structure, e.g. a catheter, can be properly placed by insertion in the internal jugular vein, 107, or the subclavian vein, 106, or through the connecting brachial, cephalic or radial veins, or even the inferior vena cava (IVC) and guided by ultrasound imaging or x-ray fluoroscopy to the proper position at the cavoatrial junction. Further variations, including combinations and/or alternative implementations, of the embodiments described herein can be readily obtained by one skilled in the art without burdensome and/or undue experimentation. Such variations are not to be regarded as a departure from the spirit and scope of the invention.

What is claimed is:

1. A method of measuring cardiac output of a heart by using an ultrasound apparatus comprising a first implantable transducer and a second implantable transducer disposed longitudinally, said method comprising these steps:
disposing the ultrasound apparatus in the superior vena cava (SVC) of the heart at a position juxtaposed to the ascending aorta of the heart carrying a blood flow;
exciting the first implantable transducer to produce an ultrasound beam crossing into the aorta;
receiving at the second implantable transducer a Doppler-shifted signal scattered from the blood flow in the aorta passing through the ultrasound beam; and
determining the velocity of the blood flow in the aorta based on the received Doppler-shifted signals.

2. The method of claim 1, wherein the first implantable transducer is a diffraction-grating transducer (DGT) and the second implantable transducer is a slab transducer.

3. The method of claim 1, wherein the first implantable transducer is a slab transducer and the second implantable transducer is a diffraction-grating transducer (DGT).

4. The method of claim 1, wherein the aorta of the heart has a diameter, further comprising the step of:
exciting an additional ultrasound pulse from the ultrasound apparatus for measuring the diameter of the aorta of the heart; and
determining the volume of blood flow in the aorta of the heart based on the velocity of the blood flow therein and the diameter of the aorta.

5. The method of claim 4, wherein the aorta comprising a far wall and near wall with respect to the SVC, wherein the additional ultrasound pulse produces an echo from the far wall and the near wall, and wherein the measuring of the diameter of the aorta is based on the time difference between the echo from the far wall of the aorta and the echo from the near wall of the aorta.

6. The method of claim 1, wherein the SVC has a wall, wherein the first and second implantable transducers are mounted on a carrying structure having a tip and a curved section near the tip for stabilizing the position of the ultrasound apparatus near the wall of the SVC.

7. The method of claim 1, wherein the first and second implantable transducers are mounted on a central-line and the disposing of the transducer apparatus comprises extending the central-line through a vessel selected from the group consisting of internal jugular vein, subclavian vein, cephalic connecting brachial vein, radial connecting brachial vein and inferior vena cava, so that the ultrasound apparatus is disposed at the juxtaposed position.

8. The method of claim 1, wherein the disposing of the transducer apparatus comprises holding the transducer apparatus on the wall of the SVC by a mechanical design.

9. The method of claim 1, wherein the aorta of the heart has a wall, a center and a cross section perpendicular to the wall, and the first and second implantable transducers are curved radially with respect to the center of the aorta of the heart so that the excited ultrasound beam overlaps with a portion of the cross section of the aorta.

10. A method of measuring cardiac output of a heart by using an ultrasound apparatus comprising a first implantable transducer and a second implantable transducer disposed longitudinally, said method comprising the steps of:
disposing the transducer apparatus in the superior vena cava (SVC) of the heart at a position juxtaposed to the ascending aorta of the heart carrying a blood flow with a velocity;
exciting the first implantable transducer with a first ultrasound beam at a first frequency at a first angle with respect to the transducer, where the first ultrasound beam crosses the aorta;
receiving on the second implantable transducer a first Doppler-shifted signal scattered from the blood flow in the aorta passing through the first ultrasound beam;
exciting the first implantable transducer to produce a second ultrasound beam at a second frequency at a second angle with respect to the transducer;
receiving on the second implantable transducer a second Doppler-shifted signal scattered from the blood flow in the aorta passing through the second ultrasound beam; and
determining the velocity of the blood flow in the aorta based on the received first and second Doppler-shifted signals.

11. The method of claim 10, wherein the first implantable transducer is a diffraction-grating transducer (DGT) and the second implantable transducer is a slab transducer.

12. The method of claim 10, wherein the aorta of the heart has a diameter, further comprising the step of:
exciting an additional ultrasound pulse for measuring the diameter of the aorta of the heart; and
determining the volume of blood flow in the aorta of the heart based on the velocity of the blood flow therein and the diameter of the aorta.

13. The method of claim 10, wherein the aorta comprising a far wall and near wall with respect to the SVC, wherein the additional ultrasound pulse produces an echo from the far wall and the near wall, and wherein the measuring of the diameter of the aorta is based on the time difference between the echo from the far wall of the aorta and the echo from the near wall of the aorta.

14. A method of measuring cardiac output of a heart using an ultrasound apparatus comprising an implantable emitting transducer for emitting an ultrasound beam, a first implantable receiving transducer and a second implantable receiving transducer for receiving ultrasound emissions, wherein the implantable emitting transducer is disposed longitudinally between the first and second implantable receiving transducers, said method comprising the steps of:
disposing the ultrasound apparatus into the superior vena cava (SVC) of the heart so that the ultrasound apparatus is positioned juxtaposed to the aorta of the heart;
exciting the implantable emitting transducer to produce a first and a second ultrasound beams, each at opposite angles;
receiving first and second Doppler-shifted signals at the first implantable receiving transducer and the second implantable receiving transducer, respectively, wherein the first Doppler-shifted signal and the second Doppler-shifted signal are scattered from the blood flow in the aorta passing through the first ultrasound beam and the second ultrasound beam, respectively; and
determining the velocity of the blood flow in the aorta based on the received first and second Doppler-shifted signals.

15. The method of claim 14, wherein the implantable emitting transducer is a diffraction-grating transducer (DGT) and the first and second implantable receiving transducers are slab transducers.

16. The method of claim 14, wherein the aorta of the heart has a diameter, further comprising the step of:
exciting an additional ultrasound pulse for measuring the diameter of the aorta of the heart; and
determining the volume of blood flow in the aorta of the heart based on the velocity of the blood flow therein and the diameter of the aorta.

17. The method of claim 16, wherein the aorta comprising a far wall and near wall with respect to the SVC, wherein the additional ultrasound pulse produces an echo from the far wall and the near wall, and wherein the measuring of the diameter of the aorta is based on the time difference between the echo from the far wall of the aorta and the echo from the near wall of the aorta.

18. A method of measuring cardiac output of a heart by using a pulse-echo ultrasound transducer, said method comprising these steps:
disposing the ultrasound transducer in the superior vena cava of the heart at a position juxtaposed to the ascending aorta of the heart carrying a blood flow;
exciting the ultrasound transducer to produce one or more ultrasound beams crossing into the aorta;
receiving at the ultrasound transducer Doppler-shifted signals scattered from the blood flow in the aorta passing through the one or more ultrasound beams; and
determining the velocity of the blood flow in the aorta based on the received Doppler-shifted signals.

19. The method of claim 18, wherein the exciting of the ultrasound transducer produces a single ultrasound beam.

20. The method of claim 18, wherein the exciting of the ultrasound transducer produces two ultrasound beams, each at a different angle with respect to the ultrasound transducer.

21. The method of claim 18, wherein the ultrasound transducer is mounted on a central-line and the disposing of the ultrasound transducer comprises extending the central-line through a vessel selected from the group consisting of internal jugular vein, subclavian vein, cephalic connecting brachial vein, radial connecting brachial vein and inferior vena cava, so that the ultrasound transducer is disposed at the juxtaposed position.
